(19) United States
(12) Patent Application Publication
(10) Pub. No.: US 2026/0033705 A1
(43) Pub. Date: Feb. 5, 2026

(54) MULTI-AXIS JOINT STRUCTURE FOR A MEDICAL INSTRUMENT

(71) Applicant: Intuitive Surgical Operations, Inc., Sunnyvale, CA (US)

(72) Inventor: Bruce SCHENA, Menlo Park, CA (US)

(73) Assignee: Intuitive Surgical Operations, Inc., Sunnyvale, CA (US)

(21) Appl. No.: 19/276,412

(22) Filed: Jul. 22, 2025

Related U.S. Application Data
(60) Provisional application No. 63/677,509, filed on Jul. 31, 2024.

(51) Int. Cl. A61B 1/00; A61B 34/30; A61B 34/35

(52) U.S. Cl. CPC A61B 1/00149; A61B 34/35; A61B 2034/301; A61B 2034/302

(57) ABSTRACT

A medical instrument is provided for use with surgical system. The medical instrument includes an instrument shaft, a proximal disk coupled to a distal end of the instrument shaft, and a distal disk coupled to an end effector. At least one joint disk is positioned between and in axial alignment with the proximal disk and the distal disk. A distal face of the joint disk includes an engagement protrusion, while a proximal face of the joint disk includes an engagement recess that is orthogonal to the engagement recess. The medical instrument also includes a coupling member positioned within the central passage extending between the proximal disk and the distal disk the coupling member is fixedly coupled to the proximal disk and the distal disk but has an absence of coupling with the joint disk.

### CROSS-REFERENCE TO RELATED APPLICATIONS

This patent application claims priority to and the filing date benefit of U.S. Provisional Patent Application No. 63/677,509, entitled “Multi-Axis Joint Structure for a Medical Instrument,” filed Jul. 31, 2024, the disclosure of which is incorporated herein by reference in its entirety.

### BACKGROUND

The embodiments described herein relate to surgical systems, and more specifically to teleoperated surgical systems. More particularly, the embodiments described herein relate to a multi-axis joint structure of a medical instrument.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient via a cannula to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

In some known systems, the instrument can deny the surgeon a desirable degree of flexibility in the placement of the end effector. For example, some instruments have rigid shafts that can limit access to a portion of a surgical site that is not in line with the insertion point. Accordingly, it may be desirable to use in instrument that includes a gimbal arrangement or other multi-axis joint structure. The use of a multi-axis joint structure can allow the surgical instrument to adapt to the geometry of the surgical access path. In some cases, the multi-axis joint structure can provide access to a portion of the surgical site that is not directly in line with the insertion point. Additionally, the use of the multi-axis joint structure can provide a wide range of motion, such as a degree of flexion that exceeds 90 degrees. Further, a multi-axis joint structure can provide more gradual change of direction at the wrist of the instrument then may be found with other design approaches, which can facilitate the passage and/or functioning of various cables that pass through the joint structure.

In some known systems the multi-axis joint structure is operably coupled to an actuator. The actuator can provide an actuating force to affect the position and/or orientation of the segments of the multi-axis joint structure and the end effector coupled thereto. Typically, a means of coupling the actuator forces to the joint structure runs through the elongated tube of the instrument. For example, control cables can be used to apply a tensile force on the segments of the joint structure to cause at least a portion of the joint structure to rotate relative to the elongated tube. Accordingly, the segments of known multi-axis joint structures are typically metal components formed using known metal manufacturing techniques, such as metal injection molding, to ensure the components have sufficient strength to receive both the tensile loads and loads applied to the end effector during a surgical procedure. Additionally, the metal components are typically expensive, complex components that commonly employ additional parts such as retention struts or links. As these joint structures are expensive, complex and challenging to assemble, it is typically desirable to process (e.g., clean, sterilize, and refurbish) the joint structure following an operative procedure so that the joint structure can be reused during a subsequent operative procedure. However, the complexity of the joint structure presents significant challenges to post- operative processing.

In view of the aforementioned, the art is continuously seeking new and improved multi-axis joint structures for use with a medical instrument.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, the present disclosure is directed to a medical instrument. The medical instrument includes an instrument shaft that is elongated along a longitudinal axis. The medical instrument includes a proximal disk coupled to a distal end of the instrument shaft, and a distal disk coupled to an end effector of the medical instrument. A joint disk is positioned between and in axial alignment with the proximal disk and the distal disk. The joint disk defines a portion of a central passage axially aligned with the longitudinal axis. Each of the joint disk and the proximal disk have a distal face and a proximal face. Each distal face includes an engagement protrusion, and each proximal face includes an engagement recess. Each engagement recess is sized to receive a corresponding engagement protrusion. The engagement protrusion of the joint disk has a length that is orthogonal to a length of engagement recess of the joint disk. The medical instrument also includes a coupling member within the central passage and extending between the proximal disk and the distal disk, the coupling member being fixedly coupled to the proximal disk and to the distal disk, the coupling member having an absence of coupling with the joint disk.

These and other features, aspects and advantages of the present invention will become better understood with reference to the following description and drawings.

### BRIEF DESCRIPTION OF THE DRAWINGS

FIG. 1 is a simplified perspective view of a teleoperated surgical system according to an embodiment with a mechanically actuated surgical instrument inserted through a port in a patient's abdomen.

FIG. 2 is a is a side view of a surgical instrument for use with teleoperated surgical system of FIG. 1.

FIG. 3 is a perspective view of a multi-axis joint structure of a medical instrument according to an embodiment.

FIG. 4 is a side view of the multi-axis joint structure of FIG. 3 in a flexed configuration.

FIG. 5 is an exploded view of the multi-axis joint structure of FIG. 3.

FIG. 6 is a cross-sectional view of the multi-axis joint structure of FIG. 3 taken at x₁-x₁.

FIGS. 7A and 7B are perspective views of a proximal face and a distal face respectively of a joint disk of the multi-axis joint structure of FIG. 3.

FIG. 8 is a planform view of the distal face of the joint disk of FIGS. 7A and 7B.

FIG. 9 is a planform view of the proximal face of the joint disk of FIGS. 7A and 7B.

FIG. 10 is a perspective view of the joint disk of FIGS. 7A and 7B depicting a distal reference plane and a proximal reference plane.

FIG. 11 is a cross-sectional view of the joint disk of FIG. 10 taken at line x₂-x₂ in FIG. 8.

FIG. 12 is a cross-sectional view of the joint disk of FIG. 10 taken at x₃-x₃ in FIG. 8.

FIG. 13 is a side view of a pair of joint disks of the multi-axis joint structure of FIG. 3 in a neutral configuration with an engagement protrusion received by an engagement recess and with the coupling member hidden for clarity.

FIG. 14 is a cross-sectional view of the pair of joint disks of FIG. 13 taken at x₄-x₄ with the coupling member depicted.

FIG. 15 is a cross-sectional view of the pair of joint disks of FIG. 13 taken at x₄-x₄ in a flexed configuration with the coupling member hidden for clarity.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Generally, the present disclosure is directed to a multi-axis joint structure for a medical instrument. The medical instrument can, for example, have an instrument shaft with a maximum outer diameter of 5.5 mm or less. The multi-axis joint structure described herein can be included in an instrument that is low-cost, easy-to-assemble, single-use, disposable, and/or size restricted. As a single-use instrument, costs associated with postoperative processing of the instrument are eliminated, thus reducing the per-procedure costs associated with the instrument. For example, a medical instrument can be a single-use gastrointestinal endoscope that is disposed of following each procedure. In view of the single-use nature instrument, it is desirable that costs associated with multi-axis joint structure be minimized. To that end, the features of the components of the joint structure described herein facilitate a multi-axis joint structure that can use very low cost molded plastic components, in lieu of complex metal components, that have sufficient strength to withstand the loads imparted by control cables and/or imparted to the end effector during a surgical procedure. Additionally, the features and arrangements of the components of the multi-axis joint structure facilitate at least a partially automated assembly process, which further lowers costs associated with the instrument.

As described herein, the multi-axis joint structure can include a proximal disk coupled to a distal end of the instrument shaft, a distal disk coupled to an end effector, and a disk stack (which can include one or more identical joint disks) positioned therebetween. The disk stack can be arranged as a single stack to form an elbow or wrist having two degrees of freedom (e.g., a double bend direction). However, in some embodiments, the disk stack can be arranged as a double stack separated by a rigid section to create an elbow-wrist assembly with four degrees of freedom. Each disk stack is constrained by two rigid end sections (e.g., the proximal disk and the distal disk) that interface to the proximal and distal portions of the instrument (e.g., the instrument shaft and the end effector), where the number of identical stacking disks (i.e., joint disks) in between can vary, providing an easily customizable range of motion for the multi-axis joint structure. Each joint disk and the proximal disk include a distal face, while each joint disk and the distal disk include a proximal face. Although the features described herein are described with reference to the proximal faces and the distal faces, it should be appreciated that the orientation of the components can be longitudinally reversed. In other words, the first structures described as being included with the proximal face and the second structures described as being included with the distal face such that the first structures can be included with the distal face while the second structures are included with the proximal face.

The joint disks, which can be identical, can be produced by many methods including machining, additive manufacturing, or injection molding from a variety of materials depending on the application requirements. For example, the joint disks can be formed from Polyoxymethylene (POM) or carbon-filled polyether ether ketone (PEEK) or other polymers having a similar inherently low coefficient of friction. The joint disks can have a circular planform. However, the joint disks can also be formed to have a non-circular planform, such as a rectilinear planform, a triangular planform, an oval planform, or an irregular planform.

The multi-axis joint structure also includes a central passage defined by the proximal disk, the joint disk, and the distal disk. The central passage is sized to receive a coupling member that extends between the proximal disk and the distal disk. The coupling member is fixedly coupled to the proximal disk and to the distal disk but has an absence of coupling with the joint disk. The coupling member can, for example, be a spine constructed from a nickel titanium alloy (e.g., nitinol) having appropriate flexibility to enforce a near-constant bending radius in the multi-axis joint structure and may provide a compressive mechanical preload to the structure. As the one or more joint disks are not directly coupled to each other nor to the proximal disk and the distal disk by joints, pins or direct fasteners, the compressive mechanical preload provided by the coupling member precludes separation of the components from the stacked arrangement.

Tension members (e.g., control cables) made of tungsten, stainless steel, and/or plastic polymers such as high-density polyethylene (HDPE), or polyphenylene benzobisoxazole (PBO) can be routed through an outer array of holes of the joint disk. Approximately diametrically opposite pairs of cables can then be used antagonistically to actuate the multi-axis joint structure throughout the range of motion.

In some embodiments, each pair of joint disks can allow motion of approximately ±20 degrees of single-axis pivot motion (e.g., pitch, or yaw) due to an interlocking of an engagement protrusion of the distal face of one joint disk with an engagement recess of a proximal face of the adjacent second joint disk. Each of the engagement protrusion and the engagement recess can be formed as cylindrical segments. In some embodiments, the engagement protrusion and the engagement recess can be split into two or more functional sections if required by the design. For any one of the identical joint disks, the engagement protrusion of the distal face is rotated 90degrees relative to the engagement recess of the proximal face. As such, the joint disks in a stacked arrangement are each clocked 90 degrees relative to each adjacent joint disk. Adjacent disks are also designed to touch each other at the extremes of motion, creating robust mechanical motion limit stops. It should be appreciated that, in some embodiments, the positioning of the engagement protrusion and the engagement recess can be reversed so that an engagement protrusion of the proximal face of one joint disk with an engagement recess of the distal face of the adjacent second joint disk.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation defines the body's pose (e.g., a kinematic pose).

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if it possesses the ability to absorb energy when it is deformed elastically, and then releases the stored energy upon unloading (i.e., returning to its original state). Many “rigid” objects have a slight inherent resilient “bendiness” due to material properties, although such objects are not considered “flexible” as the term is used herein.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Inventive aspects are described with reference to a teleoperated surgical system. An example architecture of such a teleoperated surgical system is the da Vinci® surgical system commercialized by Intuitive Surgical, Inc., Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

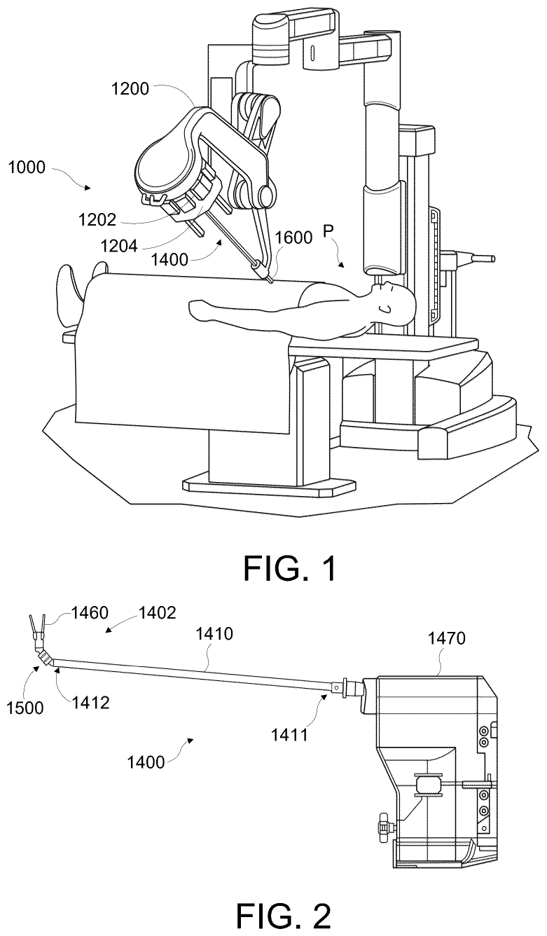

FIG. 1 is a simplified diagrammatic perspective view of a teleoperated surgical system 1000. The system 1000 includes a support assembly 1200 mounted to or near an operating table supporting a patient's body P. The support assembly 1200 supports one or more surgical instruments 1400 that operate on a surgical site within the patient's body P.

The simplified perspective view of the system 1000 shows only a single instrument 1400 to allow aspects of the invention to be more clearly seen. A functional teleoperated surgical system can further include a vision system that enables the operator to view the surgical site from outside the patient's body P. The vision system can include a video monitor for displaying images received by an optical device provided at a distal end of one of the surgical instruments 1400. The optical device can include a lens coupled to an optical fiber which carries the detected images to an imaging sensor (e.g., a CCD or CMOS sensor) outside of the patient's body P. Alternatively, the imaging sensor may be provided at the distal end of the surgical instrument 1400, and the signals produced by the sensor are transmitted along a lead or wirelessly for display on the monitor. An illustrative monitor is the stereoscopic display on the surgeon's cart in the da Vinci® Surgical System, marketed by Intuitive Surgical, Inc., of Sunnyvale, Calif.

A functional teleoperated surgical system can further include a control system for controlling the insertion and articulation of the surgical instruments 1400. This control may be effectuated in a variety of ways, depending on the degree of control desired, the size of the surgical assembly, and other factors. In some embodiments, the control system includes one or more manually operated input devices, such as a joystick, exoskeletal glove, or the like. These input devices control motors, such as servo motors, which, in turn, control the articulation of the surgical assembly. The forces generated by the motors are transferred via drivetrain mechanisms, which transmit the forces from the motors generated outside the patient's body P through an intermediate portion of the elongate surgical instrument 1400 to a portion of the surgical instrument inside the patient's body P distal from the motor.

The surgical instrument 1400 is shown inserted through an entry guide 1600, e.g., a cannula in the patient's abdomen. The entry guide 1600 is mounted onto an entry guide manipulator, which includes a mechanically actuated positioning system for positioning the distal end of the entry guide 1600 at the desired target surgical site. The mechanically actuated positioning system can be provided in a variety of forms, such as a serial link arm having multiple degrees of freedom (e.g., six degrees of freedom) or a jointed arm that provides a remote center of motion (due to either hardware or software constraints) and that is positioned by one or more unpowered, lockable setup joints mounted onto a base. Alternatively, the entry guide manipulator can be manually maneuvered so as to position the entry guide 1600 in the desired location. In some telesurgical embodiments, the input devices that control the manipulator(s) can be provided at a location remote from the patient (outside the room in which the patient is placed). The input signals from the input devices are then transmitted to the control system, which, in turn, manipulates at least one manipulator 1202 (e.g., an instrument manipulator) in response to the signals. The instrument manipulator 1202 can be coupled to the entry guide manipulator such that the instrument manipulator 1202 moves in conjunction with the entry guide 1600.

The surgical instrument 1400 is detachably connected to the mechanically actuated instrument manipulator 1202. The mechanically actuated manipulator includes a coupler 1204 to transfer controller motion from the mechanically actuated manipulator to the surgical instrument 1400. The instrument manipulator 1202 may provide a number of controller motions which the surgical instrument 1400 may translate into a variety of movements of the end effector on the surgical instrument such that the input provided by a surgeon through the control system is translated into a corresponding action by the surgical instrument.

FIG. 2 is a side view of the surgical instrument 1400 for use with the system 1000. The instrument 1400 includes a distal end portion 1402 and a proximal mechanical structure 1470. The proximal mechanical structure 1470 is coupled to a proximal and 1411 of an instrument shaft 1410. The instrument shaft 1410 can be a rigid unitary structure, a flexible unitary structure, a multi-component rigid structure, a multi-component flexible structure, or combinations thereof. The distal end portion 1402 can include an end effector 1460 coupled to a distal end 1412 of the instrument shaft 1410 via a wrist assembly 1500 (e.g., a multi-axis joint structure). The end effector 1460 can include any of a variety of surgical devices such as the forceps, a needle driver, a cautery device, a cutting tool, an imaging device (e.g., an endoscope or ultrasound probe), or a combined device that includes a combination of two or more various tools and imaging devices. The wrist assembly 1500 facilitates the orientation of the end effector 1460 relative to the target of the surgical procedure within the surgical site.

The instrument 1400 can include a set of tension members (e.g., control cables) (not shown), that can be used to affect the positioning of portions of the instrument 1400. The tension members operably couple the proximal mechanical structure 1470 to the wrist assembly 1500 and the end effector 1460. Accordingly, the tension members provide a means of transmitting forces to the wrist assembly 1500 and the end effector 1460 that is compact and flexible. The tension members are routed through the instrument shaft 1410, which can have a limited maximal outer diameter, such as a maximal outer diameter of less than 6.5 millimeters. In some embodiments, the maximal outer diameter of the shaft 1410, the wrist assembly 1500, and any of the multi-axis joint structures described herein can be less than 5.5 millimeters. In some embodiments, the maximal outer diameter of the shaft 1410, the wrist assembly 1500, and any of the multi-axis joint structures described herein can be between about 3 millimeters and 5.5 millimeters. The diminutive scale of the mechanisms in the surgical instrument 1400 creates unique mechanical conditions and issues with the construction of these mechanisms that are unlike those found in similar mechanisms constructed at a larger scale because forces and strengths of materials do not scale at the same rate as the size of the mechanisms. For example, the cables must fit within the instrument shaft 1410 and be able to bend as they pass through the joints of the wrist assembly 1500. Similarly, it is desirable to construct the wrist assembly 1500 in the manner described herein to provide sufficient strength to resist the forces applied to the wrist assembly 1500 and/or the end effector 1460 by the tension members.

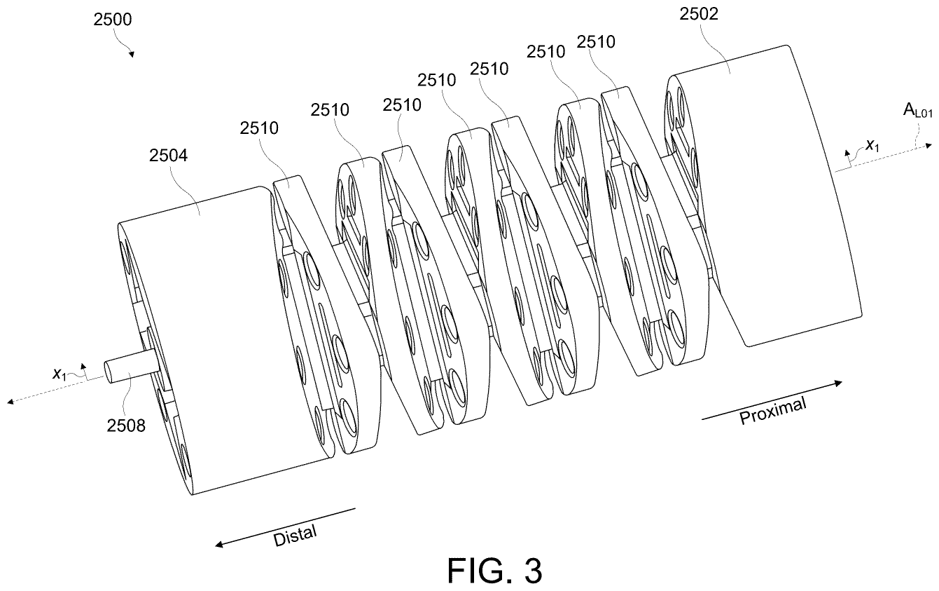

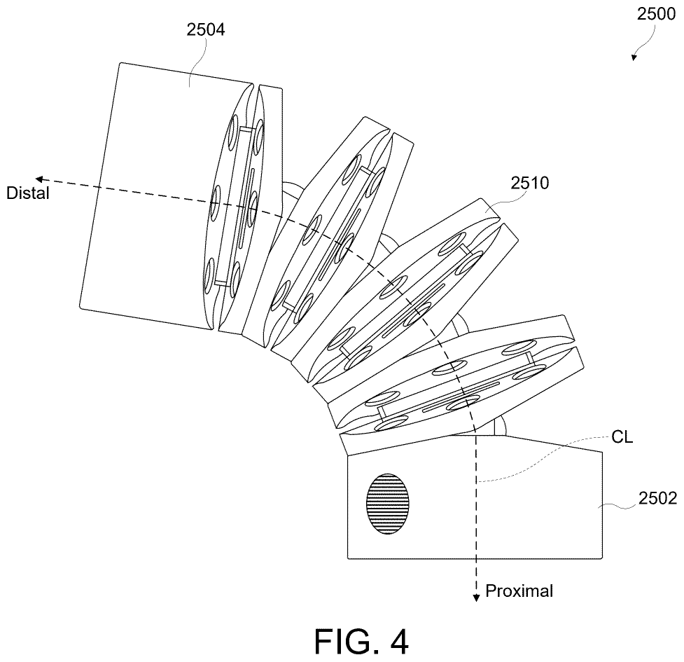

In some embodiments, the wrist assembly and/or an elbow assembly of the instrument 1400 can be a multi-axis joint structure 2500 as depicted FIGS. 3-15. Accordingly, the end effector 1460 is coupled to the instrument shaft 1410 via the multi-axis joint structure 2500. The instrument shaft 1410 defines a longitudinal axis A_{LO1} for the instrument 1400. In a neutral configuration (as depicted in FIG. 3), the multi-axis joint structure 2500 is axially aligned with the longitudinal axis A_{LO1}. In a flexed configuration (as depicted in FIG. 4), at least a portion of the multi-axis joint structure 2500 is not aligned with the longitudinal axis A_{LO1}. Similarly stated, in a flexed configuration a center line CL of the multi-axis joint structure 2500 (which can be curved) is offset from the longitudinal axis A_{LO1}.

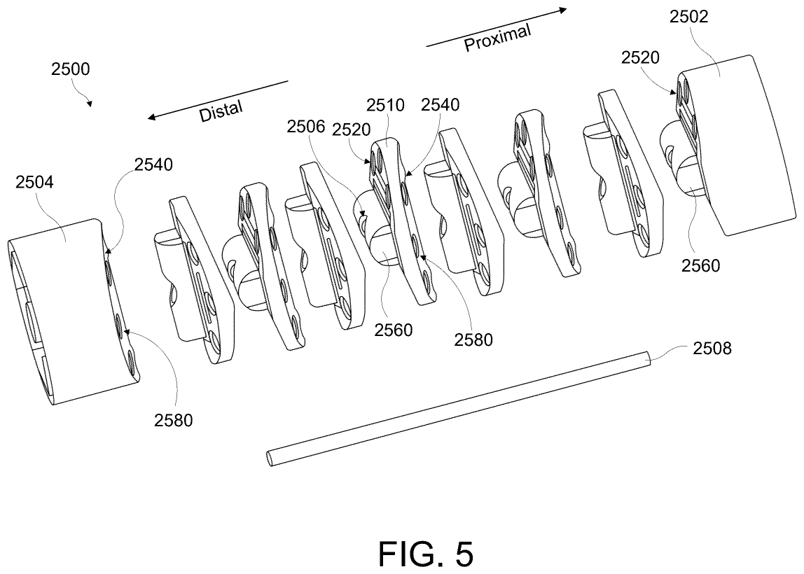

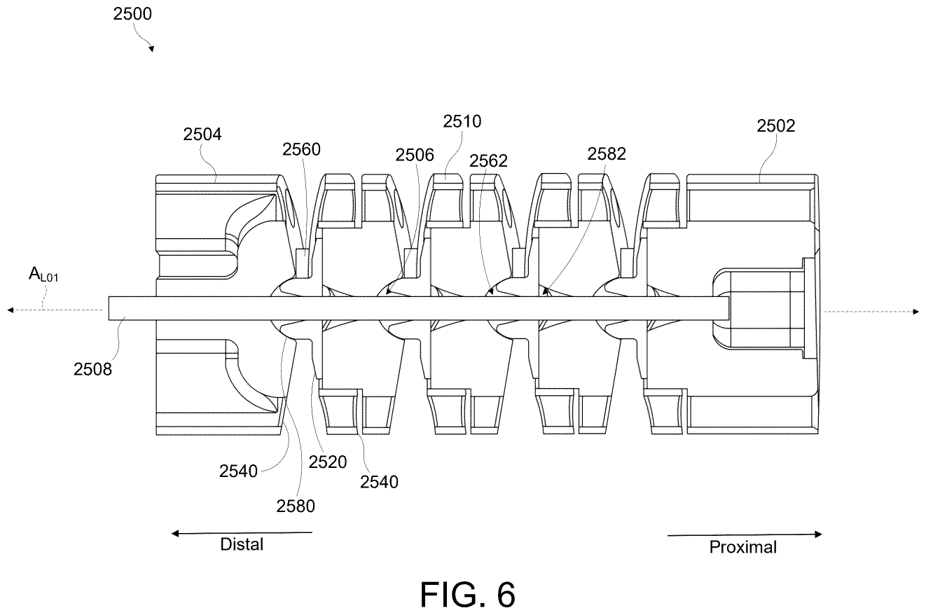

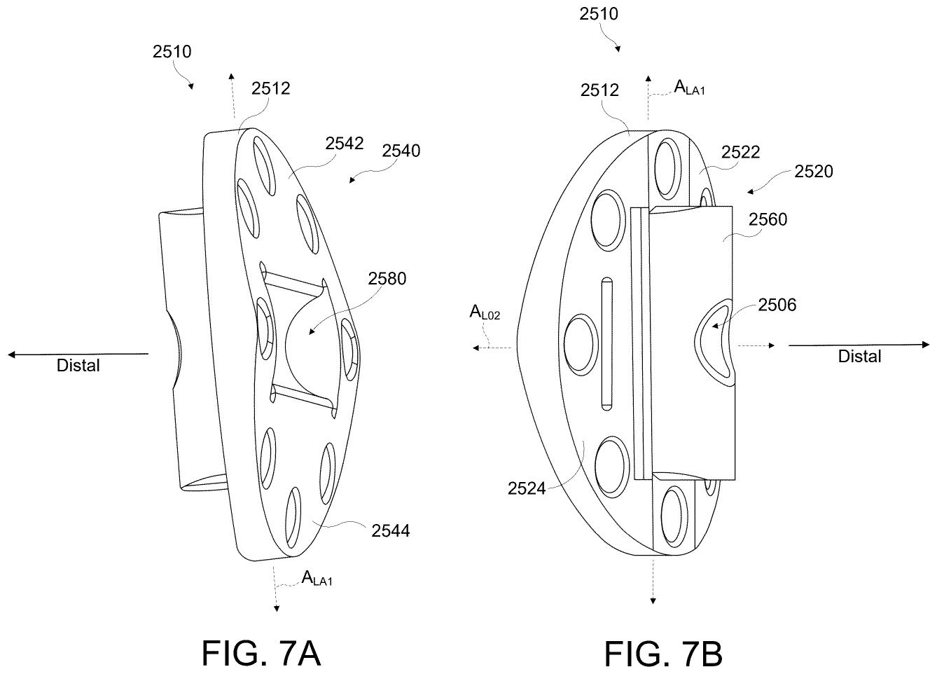

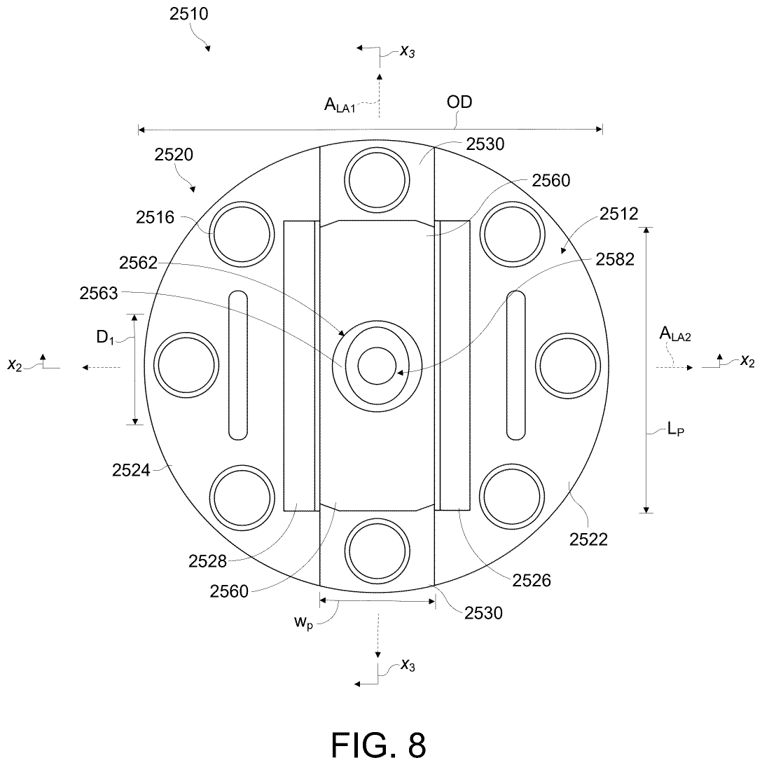

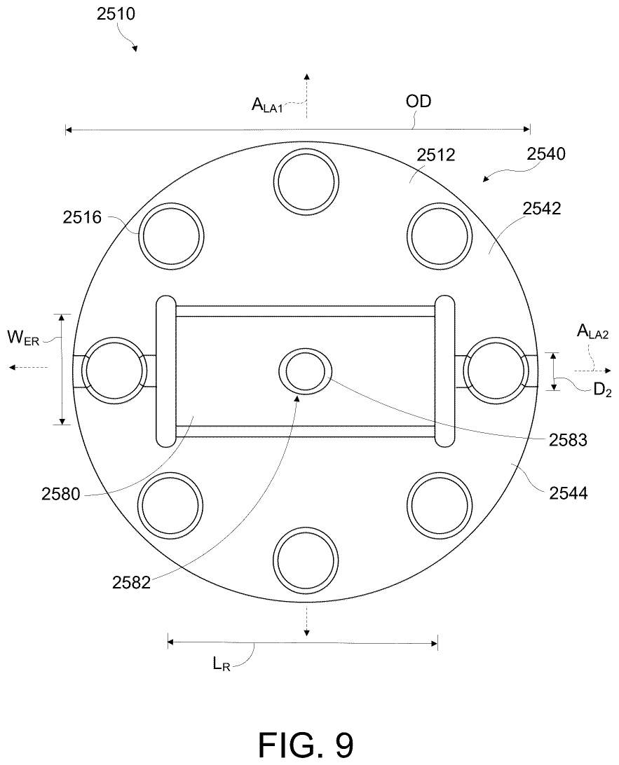

The multi-axis joint structure 2500 includes a proximal disk 2502 coupled to the distal end 1412 of the instrument shaft 1410 and a distal disk 2504 coupled to the end effector 1460 (or any other suitable end effector) of the medical instrument 1400. At least one joint disk 2510 is positioned between and in alignment with the proximal disk 2502 and the distal disk 2504. Specifically, the at least one joint disk 2510 is aligned with the proximal disk 2502 and the distal disk 2504 along the center line CL. Additionally, the at least one joint disk 2510 is aligned with the proximal disk 2502 and the distal disk 2504 along the longitudinal axis A_{LO1} when the multi-axis joint structure 2500 is in the neutral configuration. In some embodiments, the joint disk 2510 is one of a set of joint disks in a stacked arrangement. However, since each joint disk 2510 of the set of joint disks is identical in this embodiment, the following description references a single joint disk 2510, unless specified otherwise, with the description applying equally to any joint disk of the set of joint disks. The joint disk 2510 defines a portion of a central passage 2506 that is axially aligned with the longitudinal axis A_{LO1} on a condition that the multi-axis joint structure 2500 is in the neutral configuration. Each of the joint disks 2510 and the proximal disk 2502 have a distal face 2520 (FIG. 5), as further described herein. Similarly, each of the joint disks 2510 and the distal disk 2504 have a proximal face 2540, as further described herein. Each distal face 2520 is identical to every other distal face 2520 of the multi-axis joint structure 2500. The proximal face 2540 is identical to every other proximal face 2540 of the multi-axis joint structure 2500. As depicted in FIG. 5, each distal face 2520 includes an engagement protrusion 2560. Each proximal face 2540 includes an engagement recess 2580. As depicted in FIG. 6, each engagement recess 2580 is sized to receive a corresponding engagement protrusion 2560 but has an absence of retention coupling (e.g., direct coupling) therewith. Similarly stated, when the protrusion 2560 of a first joint disk 2510 is matingly disposed within the engagement recess 2580 of a second (adjacent) joint disk 2510, the joint disks themselves to not provide for a captive coupling. Rather, the two joint disks 2510 are maintained in mating contact via additional structures (e.g., the coupling member 2508 or via a tensile force applied to the distal disk 2504 and/or end effector 1460 via the tension members that are routed through the joint structure). As depicted in FIG. 7A, the engagement protrusion 2560 of the joint disk 2510 has a length L_{P} (FIG. 8) that is orthogonal to a length L_{R} (FIG. 9) of the engagement recess 2580 of the joint disk 2510. Said another way, a longitudinal axis of the engagement protrusion 2560 is orthogonal to a longitudinal axis of the engagement recess 2580. In this manner, each joint disk 2510 can be oriented 90 degrees from its adjacent joint disk about the longitudinal axis A_{LO1}. It should be appreciated that that the structures and features of each proximal face and the structures and features of each distal face for the multi-axis joint structure 2500 can be longitudinally reversed. Accordingly, the features and structures described herein with reference to each distal face can, in some embodiments, be positioned in reference to each proximal face and vice versa. For example, in some embodiments, each distal face 2520 can include the engagement recess 2580, while each proximal face 2540 includes the engagement protrusion 2560.

As depicted in FIG. 6, a coupling member 2508 is positioned within a central passage 2506 defined, at least in part, by each joint disk 2510 within the multi-axis joint structure 2500. The coupling member 2508 extends between the proximal disk 2502 and the distal disk 2504. The coupling member 2508 is fixedly coupled to the proximal disk 2502 and to the distal disk 2504. The coupling member is routed through the central passage 2506 of the set of joint disks 2510 such that each joint disk 2510 can move relative to the coupling member 2508. In this manner, the joint disks 2510 can rotate slightly relative to the coupling member 2508 when the joint assembly 2500 is moved from the neutral configuration towards a flexed configuration. The absence of a fixed coupling between each joint disk 2510 and the coupling member 2508 also facilitates assembly of the joint assembly 2500 by allowing the joint disks 2510 to be threaded onto the coupling member 2508.

The coupling member 2508 (which functions as a spine member) has the appropriate flexibility (or stiffness) to facilitate a near-constant bending radius in the multi-axis joint structure 2500. On a condition that the flexibility of the coupling member 2508 is greater than is desirable, the multi-axis joint structure 2500 may manifest an unequal rotation between the components. On a condition that the flexibility of the coupling member 2508 is less than is desirable, an excessive amount of tensile force may be required from the tension members to affect the position of the multi-axis joint structure 2500. Accordingly, the diameter of the coupling member 2508 is between 0.35 mm and 0.45 mm (e.g., 0.41 mm). In some embodiments, the coupling member 2508 is an alloy of nickel and titanium, such as nitinol, or other super elastic material. The nickel-titanium alloy may not yield at high strain and can accommodate high bending without failure. In some embodiments, the coupling member 2508 can provide a compressive mechanical preload to the multi-axis joint structure 2500. Accordingly, the coupling member 2508 can provide an axial spring-load to facilitate torsional stiffness of the multi-axis joint structure 2500. Said another way, insofar as each engagement protrusion 2560 can be received by a corresponding engagement recess 2580 but is not retentively coupled thereto, the compressive mechanical preload of the coupling member 2508 can maintain the multi-axis joint structure 2500 in the stacked arrangement.

FIGS. 7A-12 depict multiple views of a single joint disk 2510 of a set of substantially identical joint disks of the multi-axis joint structure 2500. The joint disk 2510 includes a circular body 2512. The circular body 2512 is positioned orthogonal to the longitudinal axis A_{LO2} of the joint disk 2510, which is coaxial with the longitudinal axis A_{LO1} defined by the instrument shaft 1410 on a condition that the multi-axis joint structure 2500 is in the neutral position depicted in FIGS. 3 and 6. The circular body 2512 defines a portion of the central passage 2506. The central passage 2506 defined by the circular body 2512 is coaxial with the longitudinal axis A_{LO2} of the joint disk 2510 and is configured to receive the coupling member 2508 (FIG. 5).

FIGS. 7A and 7B depict two perspective views of the same joint disk 2510. The view of FIG. 7A is from a proximal position viewing in the distal direction, while the view of FIG. 7B is from a distal position viewing in the proximal direction. Similarly, FIGS. 8 and 9 are two planform views of the same joint disk 2510, with FIG. 8 depicting a distal face 2520 and FIG. 9 depicting a proximal face 2540. As depicted in FIGS. 7B and 8, the joint disk 2510 includes the distal face 2520 of the circular body 2512. The distal face 2520 includes a first distal contact face 2522, a second distal contact face 2524, and the engagement protrusion 2560. The engagement protrusion 2560 is positioned between the first distal contact face 2522 and the second distal contact face 2524. Each of the first distal contact face 2522 and the second distal contact face 2524 can cover at least 35 percent of the surface area of the distal face 2520. In some embodiments, the engagement protrusion 2560 is formed as a cylindrical segment (e.g., a convex cylindrical segment) that has a length L_{P} (FIG. 8) bisected by the longitudinal axis A_{LO2} of the joint disk 2510. The length L_{P} of the engagement protrusion 2560 (i.e., of the cylindrical segment) extends parallel to a first lateral axis A_{LA1}. The first lateral axis A_{LA1} is orthogonal to the longitudinal axis A_{LO2} of the joint disk 2510 and to a second lateral axis A_{LA2} (FIG. 8).

As depicted in FIG. 7A and 9, the joint disk 2510 includes the proximal face 2540 of the circular body 2512. The proximal face 2540 of the joint disk 2510 is positioned longitudinally opposite the distal face 2520 of the joint disk 2510. The proximal face 2540 includes a first proximal contact face 2542, a second proximal contact face 2544, and an engagement recess 2580. The engagement recess 2580 is positioned between the first proximal contact face 2542 and the second proximal contact face 2544. Each of the first proximal contact face 2542 and the second proximal contact face 2544 can cover at least 35 percent of the surface area of the proximal face 2540. Like the engagement protrusion 2560, in some embodiments the engagement recess 2580 is formed as a cylindrical segment (e.g., a concave cylindrical segment). The engagement recess 2580 has a length L_{R} that is bisected by the longitudinal axis A_{LO2} of the joint disk 2510. The length L_{R} of the engagement recess 2580 (i.e., of the cylindrical segment) extends parallel to the second lateral axis A_{LA2} orthogonal to the first lateral axis A_{LA1} and the longitudinal axis A_{LO2} of the joint disk 2510. On a condition that the engagement protrusion 2560 is received by the engagement recess 2580 of a distally adjacent component, such as an adjacent joint disk 2510, the engagement protrusion 2560 and the engagement recess 2580 form a pivot about which the joint disk 2510 can rotate relative to the distally adjacent component (e.g., another joint disk 2510).

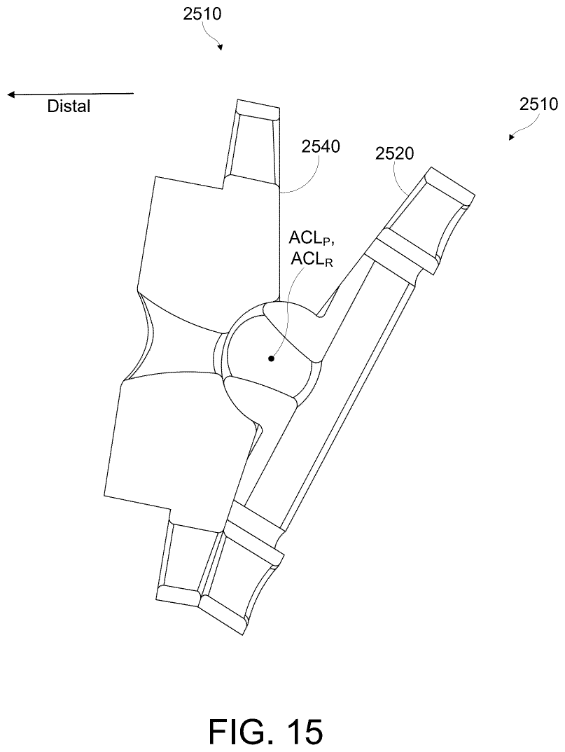

In some embodiments, the positioning of the first distal contact face 2522, the second distal contact face 2524, the first proximal contact face 2542, and/or the second proximal contact face 2544 can define a range of motion limit between the joint disk 2510 and a longitudinally adjacent component of the multi-axis joint structure 2500, such as the proximal disk 2502, the distal disk 2504, and/or an additional joint disk 2510 of the set of joint disks. The range of motion limit can correspond to the range of pivot motion about the pivot axis defined by the engagement protrusion 2560 and the engagement recess 2580 that brings a distal contact face into contact with a facing proximal contact face as depicted in FIG. 15. The contact between the opposing contact faces precludes further rotation about the pivot axis. Said another way, the first distal contact face 2522 can be in contact with an opposing first proximal contact face 2544 on a condition that the proximal disk 2502 and the joint disk 2510, the distal disk and the joint disk, and/or the joint disk and a longitudinally adjacent point disk are in a position of maximum flexion as depicted in FIG. 15.

In some embodiments, the engagement protrusion 2560 has a width W_{P} that extends parallel to the second lateral axis A_{LA2} orthogonal to the length L_{P} of the engagement protrusion 2560, which is greater than the width W_{P}. The width W_{P} can be bisected by the longitudinal axis A_{LO2} of the joint disk 2510. Said another way, the engagement protrusion 2560 can be centered about the longitudinal axis A_{LO2} of the joint disk 2510 with the longitudinal axis A_{LO2} bisecting both the length L_{P} and the width W_{P} of the engagement protrusion 2560. Similarly, in some embodiments, the engagement recess 2580 has a width W_{ER} that extends parallel to the first lateral axis A_{LA1} orthogonal to the length L_{R} of the engagement recess 2580, which is greater than the width W_{ER}. Like the engagement protrusion 2560, the width W_{ER} of the engagement recess 2580 can be bisected by the longitudinal axis A_{LO2} of the joint disk 2510. Accordingly, the engagement recess 2580 can be centered about the longitudinal axis A_{LO2} of the joint disk 2510 with the longitudinal axis A_{LO2} bisecting both the length L_{R} and the width W_{ER} of the engagement recess 2580. In order to receive the engagement protrusion 2560, the length L_{R} and the width W_{ER} of the engagement recess 2580 are greater than the length L_{P} and the width W_{P} respectively of the engagement protrusion 2560. Additionally, each engagement protrusion 2560 is configured to resist a torque load about the longitudinal axis A_{LO1} of the multi-axis joint structure 2500 on a condition that the engagement protrusion 2560 is received by the corresponding engagement recess 2580. In other words, the length L_{P} of the engagement protrusion 2560 being greater than the width W_{P} of the engagement protrusion 2560 (e.g., the engagement protrusion 2560 being formed as a cylindrical segment rather than a spherical segment) precludes rotation of the engagement protrusion 2560, and thus the joint disk 2510, about the longitudinal axis A_{LO2} of the joint disk 2510 on a condition that the engagement protrusion 2560 is received by the corresponding engagement recess 2580. It should be appreciated that the compressive mechanical preload provided by the coupling member 2508 precludes the separation of the engagement protrusion 2560 from the engagement recess 2580 in response to a torsional load and, thus, facilitates the preclusion of the rotation of the joint disk about the longitudinal axis A_{LO2} of the joint disk 2510 relative to the other components of the multi-axis joint structure 2500 described herein.

In some embodiments in which joint disk 2510 is one of a set of joint disks positioned in axial alignment between the proximal disk 2502 and the distal disk 2504, as depicted in FIG. 5, each joint disk 2510 is clocked by 90 degrees relative to each adjacent joint disk 2510. In other words, the first joint disk is rotated relative to a longitudinally adjacent joint disk so that the first lateral axis A_{LA1} of the first joint disk is brought into parallel alignment with the second lateral axis A_{LA2} of the longitudinally adjacent joint disk thereby aligning the engagement protrusion 2560 of the first joint disk with the engagement recess 2580 of the longitudinally adjacent joint disk or vice versa. Accordingly, the joint disk 2510 is arranged to pivot only about the first lateral axis A_{LA1} of the joint disk 2510 relative to a distally adjacent joint disk of the set of joint disks or the distal disk 2504. The joint disk 2510 is arranged to pivot only about the second lateral axis A_{LA2} of the joint disk 2510 relative to a proximately adjacent joint disk of the set of joint disks or the proximal disk 2502.

As depicted in FIG. 8, in some embodiments, the joint disk 2510 (i.e., the circular body 2512) has a maximal outer diameter OD that is orthogonal to the longitudinal axis A_{LO2} of the joint disk 2510. In such embodiments, the length L_{P} of the engagement protrusion 2560 has a ratio relative to the maximal outer diameter OD of between 1:1.5 and 1:1.7 (e.g., between 1:1.53 and 1:1.68). Said another way, in some embodiments the length L_{P} is at least 60 percent of the maximal outer diameter OD. In some embodiments, the length L_{P} is between 62 and 65 percent of the maximal outer diameter OD. In some embodiments the length L_{P} is less than 70 and more than 63 percent of the maximal diameter OD. Forming the engagement protrusion 2560, and the corresponding engagement recess 2580, to extend across at least 60% of the maximal outer diameter OD of the joint disk 2510 (i.e., of the circular body 2512) increases the loadbearing capacity of the joint disk 2510 relative to engagement protrusion of a shorter length. This increased loadbearing capacity derived from the size of the engagement protrusion 2560 facilitates forming the joint disk 2510 from a polymer without compromising the function of the multi-axis joint structure 2500. In contrast, a correspondingly smaller (e.g., shorter) engagement protrusion would necessitate forming the joint disk from a material having a higher loadbearing capacity, such as the metal injection molded joint disks of known systems. Therefore, the increased loadbearing capacity of the joint disk 2510 resulting from the size and shape of the engagement protrusion 2560 described herein facilitates the production of low-cost, single-use joint structures.

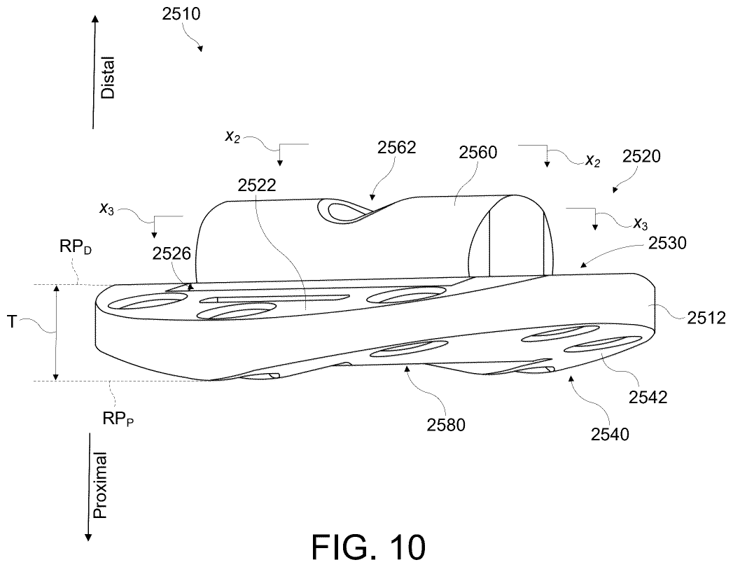

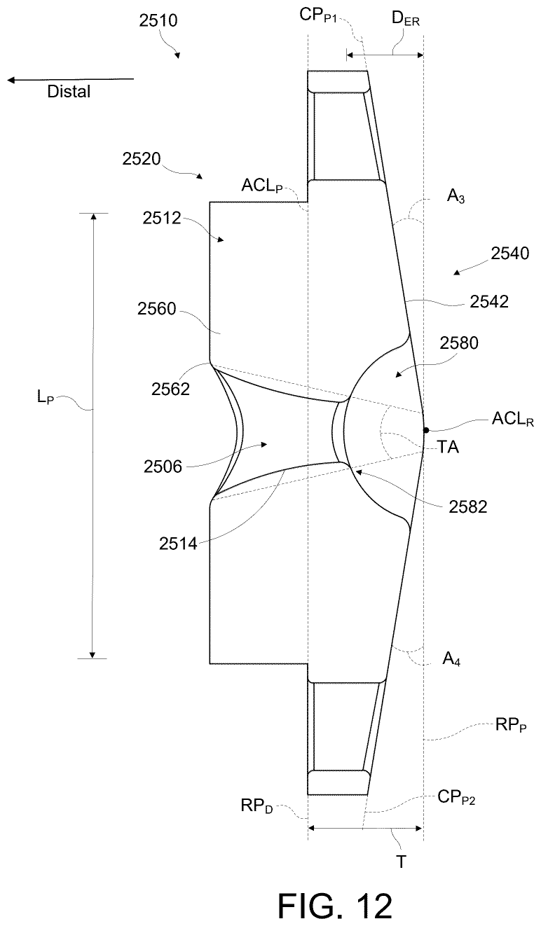

In some embodiments, the loadbearing capacity of the joint disk 2510 can be affected by a ratio of the thickness T of the joint disk 2510 to the outer diameter OD and/or by a ratio of the depth D_{ER} of the engagement recess 2580 relative to the thickness T. To that end, the joint disk 2510 can define a distal reference plane RP_{D} (FIG. 10) that is orthogonal to the longitudinal axis A_{LO2} of the joint disk 2510. The engagement protrusion 2560 extends longitudinally outward, in the distal direction, from the distal reference plane RP_{D}. The joint disk 2510 also defines a proximal reference plane RP_{P} (FIG. 10) that is similarly orthogonal to the longitudinal axis A_{LO2} of the joint disk 2510 and parallel to the distal reference plane RP_{D}. The longitudinal separation between the distal reference plane RP_{D} and the proximal reference plane RP_{P} corresponds to a maximal thickness T of the joint disk 2510 (i.e., of the circular body 2512). In some embodiments, the thickness T has a ratio relative to the maximal outer diameter OD of between 1:5.2 and 1:5.8 (e.g., 1:5.5 to 1:5.7). Accordingly, the maximal thickness T for a joint disk 2510 having a maximal outer diameter of 5.5 mm is between 0.95 mm and 1.06 mm. In some embodiments, a depth D_{ER} (FIG. 12) of the engagement recess 2580 along the longitudinal axis A_{LO2} of the joint disk 2510 relative to the proximal reference plane RP_{P} can have a ratio relative to the maximal thickness T of between 1:1.4 and 1:1.5. Said another way, the engagement recess 2580 can have a depth D_{ER} that extends from the proximal reference plane RP_{P} through at least 65 percent but less than 70 percent of the maximal thickness T of joint disk 2510. Such a depth D_{ER} of the engagement recess 2580 facilitates the receiving of the engagement protrusion 2560 to resist a torsional loading of the multi-axis joint structure 2500 and to increase the loadbearing capacity thereof, which further facilitates the forming of the joint disk 2510 from a polymer.

As depicted in FIG. 8, in some embodiments, the distal face 2520 includes a first recessed portion 2526. The first recessed portion 2526 extends parallel to the length L_{P} of the engagement protrusion 2560. The first recessed portion 2526 is positioned between the engagement protrusion 2560 in the first distal contact face 2522. The distal face 2520 can also include a second recessed portion 2528. The second recessed portion 2528 can similarly extend parallel to the length L_{P} of the engagement protrusion 2560 between the engagement protrusion 2560 and the second distal contact face 2524. Each of the first and second recessed portions 2526, 2528 can be positioned substantially parallel to the distal reference plane RP_{D}. The first recessed portion 2526 and the second recessed portion 2528 are positioned to relieve a stress concentration and/or prevent an obstruction on a condition that the joint disk 2510 is at a range of motion limit relative to an adjacent component of the multi-axis joint structure 2500. For example, the first recessed portion 2526 precludes an interference with the first distal contact face 2522 being placed in contact with an adjacent first proximal contact face 2542 on a condition that the joint disk 2510 is pivoted about the first lateral axis A_{LA1} two a range of motion limit.

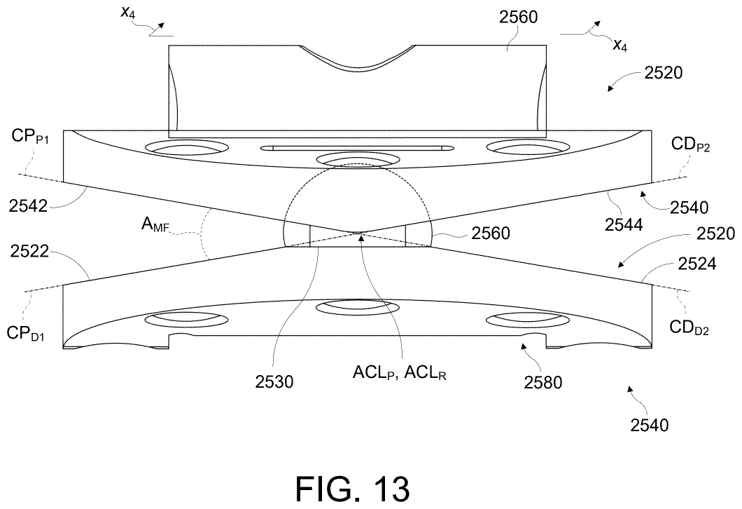

In some embodiments, the distal face 2520 includes a transition portion 2530. The transition portion 2530 extends radially outward from the engagement protrusion 2560 substantially parallel to the distal reference plane RP_{D}. In other words, the transition portion 2530 can extend radially outward from the ends of the engagement protrusion 2560 between the maximal outer diameter of the joint disk 510 and the engagement protrusion 2560. The transition portion 2530 is positioned between the first distal contact face 2522 and the second distal contact face 2524. The transition portion 2530 extends across the width W_{P} of the engagement protrusion 2560. As depicted in FIG. 13, the transition portion 2530 is shaped to preclude contact with an adjacent component of the multi-axis joint structure 2500 on a condition that the joint disk 2510 is pivoted about the first lateral axis A_{LA1} (e.g., about the engagement protrusion 2560). For example, in some embodiments, the transition portion 2530 can be a substantially planar portion of the circular body 2512 extending along the distal reference plane RP_{D}.

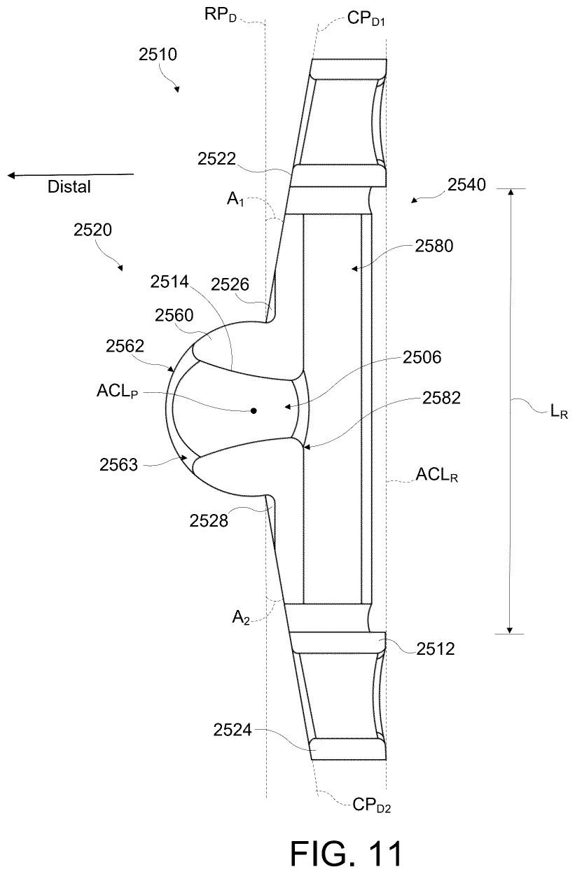

In some embodiments, the length L_{P} of the engagement protrusion 2560 extends along an axial centerline ACL_{P} (FIGS. 11 and 12) of the cylindrical segment. The axial centerline ACL_{P} can lie on the distal reference plane RP_{D}. In some embodiments, the axial centerline ACL_{P} is a pivot axis parallel to or coaxial with the first lateral axis A_{LA1} about which the joint disk 2510 can pivot relative to a distally adjacent component of the multi-axis joint structure 2500.

As depicted in FIG. 11, the first distal contact face 1522 defines a first distal contact plane CP_{D1}. The first distal contact plane CP_{D1} includes the axial centerline ACL_{P} of the engagement protrusion 2560 and is inclined relative to the distal reference plane RP_{D}. Similarly, the second distal contact face 2524 defines a second distal contact plane CP_{D2}. The second distal contact plane CP_{D2} includes the axial centerline ACL_{P}, intersects the first distal contact plane CP_{D1}, and is inclined relative to the distal reference plane RP_{D}. A magnitude of the inclination A₁ of the first distal contact plane CP_{D1} and the magnitude of the inclination A₂ of the second distal contact plane CP_{D2} are equal thereby facilitating an equal range of motion in either direction about the first lateral axis A_{LA1} (i.e., about the ACL_{P}). The inclinations A₁, A₂ of each of the first distal contact plane CP_{D1} and the second distal contact plane CP_{D2} are each equal to one half of a maximal flexion angle AMF (FIG. 13) of the joint disk 2510 relative to a distally adjacent component of the multi-axis joint structure 2500, such as the distal disk 2504 or an additional joint disk 2510 of the set of joint disks. The maximum flexion angle A_{MF} corresponds to a maximal range of motion of the joint disk 2510 when pivoting from a neutral configuration, as depicted in FIG. 13, to a fully flexed configuration (e.g., configuration of maximum flexion), in FIG. 15, in which the first or second distal contact face 1522, 1524 is in contact with the first or second proximal contact face 1542, 1544 of the distally adjacent component of the multi-axis joint structure 2500 (e.g., a distally adjacent joint disk 2510). As the first or second distal contact face 1522, 1524 is in contact with the first or second proximal contact face 1542, 1544 of the distally adjacent component in the configuration of maximum flexion, a flexion angle between the opposing faces in contact with one another has a magnitude of zero.

In some embodiments, the length L_{R} of the engagement recess 2580 extends along an axial centerline ACL_{R} (FIGS. 11 and 12) of the cylindrical segment. The axial centerline ACL_{R} can lie on the proximal reference plane RP_{P}. The axial centerline ACL_{R} can, for example, be proximal to the circular body 2512. In some embodiments, the axial centerline ACL_{R} is a pivot axis parallel to or coaxial with the second lateral axis A_{LA2} about which the joint disk 2510 can pivot relative to a proximately adjacent component of the multi-axis joint structure 2500. As depicted in FIGS. 13 and 15, in some embodiments, a longitudinal axis of the engagement recess 2580 (e.g., the axial centerline ACL_{R}) is coaxial with a longitudinal axis of the corresponding engagement protrusion 2560 (e.g., the axial centerline ACL_{P}) on a condition that the engagement protrusion 2560 is received by an adjacent engagement recess 2580 or vice versa. The coaxial longitudinal axes, thus, define a pivot axis for the joint disk 2510 relative to a longitudinally adjacent component of the multi-axis joint structure 2500.

As depicted in FIG. 12, the first proximal contact face 1542 defines a first proximal contact plane CP_{P1}. The first proximal contact plane CP_{P1} includes the axial centerline ACL_{R} of the engagement recess 2580 and is inclined relative to the proximal reference plane RP_{P}. Similarly, the second proximal contact face 2544 defines a second proximal contact plane CP_{P2}. The second proximal contact plane CP_{P2} includes the axial centerline ACL_{R}, intersects the first proximal contact plane CP_{P1}, and is inclined relative to the proximal reference plane RP_{P}. A magnitude of the inclination A₃ of the first proximal contact plane CP_{P1} and the magnitude of the inclination A₄ of the second proximal contact plane CP_{P2} are equal thereby facilitating an equal range of motion in either direction about the second lateral axis A_{LA2} (i.e., about the ACL_{R}). The inclinations A₃, A₄ of each of the first proximal contact plane CP_{P1} and the second proximal contact plane CP_{P2} are each equal to one half of a maximal flexion angle A_{MF} (FIG. 13) of the joint disk 2510 relative to a proximately adjacent component of the multi-axis joint structure 2500, such as the proximal disk 2502 or an additional joint disk 2510 of the set of joint disks. The maximum flexion angle A_{MF} corresponds to a maximal range of motion of the joint disk 2510 when pivoting from a neutral configuration, as depicted in FIG. 13, to a fully flexed configuration, in FIG. 15, in which the first or second proximal contact face 1542, 1544 is in contact with the first or second distal contact face 1522, 1524 of the proximally adjacent component of the multi-axis joint structure 2500 (e.g., a proximally adjacent joint disk 2510). In some embodiments, the inclinations A₃, A₄ of each of the first proximal contact plane CP_{P1} and the second proximal contact plane CP_{P2} and the inclinations A₁, A₂ of each of the first distal contact plane CP_{D1} and the second distal contact plane CP_{D2} are each equal to one another, with each opposing pair of inclinations defining the maximum flexion angle A_{MF}.

In some embodiments, the engagement protrusion 2560 of each distal face 2520 defines a distal passage opening 2562 of the central passage 2506. The engagement recess 2580 of each proximal face 2540 defines a proximal passage opening 2582 of the central passage 2506. In some embodiments, each of the distal passage opening 2562 and the proximal passage opening 2582 include a corresponding chamfer region and/or a rounded over region 2563, 2583 that surrounds the longitudinal axis A_{LO2} of the joint disk 2510 and/or the longitudinal axis A_{LO1} of the multi-axis joint structure 2500. A diameter D₂ of the proximal passage opening 2582 is less than a diameter D₁ of the distal passage opening 2562.

In some embodiments, a ratio of the diameter D₂ of the proximal passage opening 2582 of the joint disk 2510 to the diameter D₁ of the distal passage opening 2562 of the joint disk 2510 corresponds to a maximum range of angular motion between the joint disk 2510 and a longitudinally adjacent component of the multi-axis joint structure 2500. Said another way, the diameter D₁ of the distal passage opening 2562 is sized so that contact between the coupling member 2508 and the distal passage opening 2562 does not interfere with placing the first distal contact face 2522 or the second distal contact face 2524 in contact with a distally adjacent first proximal contact face 2542 or second proximal contact face 2544 as depicted in FIGS. 4 and 15. As depicted in FIG. 15, in some embodiments, the diameter D₂ the proximal passage opening 2582 can be sized so that the proximal passage opening 2582 and an adjacent distal passage opening 2562 are tangent to each other on a condition that opposing proximal and distal contact faces are in contact with one another.

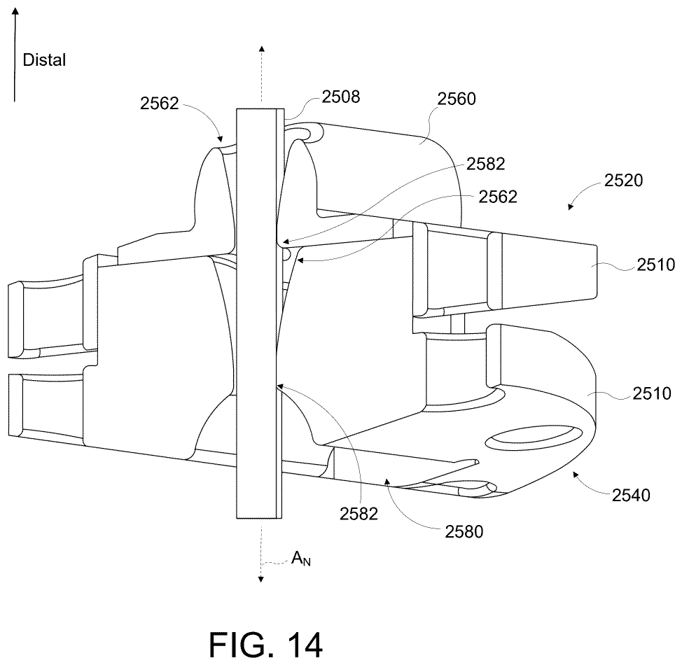

As depicted in FIG. 14, in some embodiments, each proximal passage opening 2582 is sized to maintain the coupling member 2508 in alignment with a neutral axis A_{N} extending between the proximal disk 2502 and the distal disk 2504. Said another way, the proximal passage opening 2582 can be sized so that the portion of the coupling member 2508 within the proximal passage opening 2582 is maintained centered on the neutral axis A_{N}. The portion of the coupling member 2508 corresponding to the neutral axis A_{N} has neither extension nor compression on a condition that the multi-axis joint structure 2500 is displaced from the neutral configuration depicted in FIG. 3. The diameter D₂ of the proximal passage opening 2582 can, for example, be in the range of at least 101 percent to 110 percent (e.g., 105 percent) of the diameter of the coupling member 2508.

In some embodiments, the joint disk 2510 includes a central passage wall 2514 (FIG. 12). The central passage wall 2514 extends between the proximal passage opening 2582 and the distal passage opening 2562 of the joint disk 2510. The central passage wall 2514 has a taper angle TA that extends away from the longitudinal axis A_{LO2} of the joint disk 2510 in the distal direction. The taper angle TA is proportional to a maximum range of angular motion between the joint disk 2510 and a longitudinally adjacent component of the multi-axis joint structure 2500. In some embodiments, the taper angle TA has a magnitude that is four times the magnitude of the inclination A₁ of the first distal contact plane CP_{D1} relative to the distal reference plane RP_{D}.

In some embodiments, the central passage wall 2514 extending between the proximal passage opening 2582 and the distal passage opening 2562 of the joint disk 2510 can be curvilinear in a longitudinal direction. The radius of curvature of the central passage wall 2514 in the longitudinal direction can have a magnitude that, as depicted in FIG. 15, establishes a single smooth curve extending at least between the proximal passage opening 2582 of a first joint disk 2510 and the distal passage opening 2562 of a distally adjacent joint disk 2510 on a condition that the joint disks are at a position of maximum flexion. The single smooth curve facilitates maintaining a near-constant bending radius in the multi-axis joint structure 2500, while minimizing wear on the coupling member 2508 that can result from impingements within the central passage 2506.

In some embodiments, the circular body 2512 can define a set of control passages 2516. The control passages can be distributed circumferentially about the central passage 2506. At least a portion of the control passages 2516 can be configured to receive a set of tension members (not shown) the joint disk 2510 has an absence of coupling to the tension members. Accordingly, the tension members can be moved relative to the joint disk 2510 while applying a tensile load to the distal disk 2504 and/or the end effector 1406 of the instrument 1400.

In some embodiments, the medical instrument, such as described herein can include an instrument body, an end effector, an articulatable wrist, a set of control cables, and an elongated coupling member. The instrument body can be elongated along a longitudinal axis between a distal end and a proximal end, with the end effector being coupled to the distal end. The articulatable wrist can be coupled between the instrument and the instrument body. In some embodiments, the articulatable wrist is elongated and configured to curve along a first plane, along a second plane transverse to the first plane, and in between the first and second plane without imparting rotation to the instrument body. Accordingly, the articulatable wrist can include a set of wrist links arranged in a stack, which can extend along the longitudinal axis. The set of wrist links can include a first link rotationally engaged with and laterally constrained by a second link. The set of control cables can extend along the longitudinal axis, peripherally within the set of wrist links. Said another way, the set of control cables can extend generally along the longitudinal axis but at a radial distance from an axial midline. Being thus positioned, the set of control cables are arranged to selectively apply a peripheral tension to the first link and second link to cause the articulatable wrist to curve. Said another way, the set of control cables can be positioned to apply a tension to the outer part of the link that varies based on a circumferential position of each of the control cables. This nonuniformly applied tension causes rotation about the link axes. Additionally, the elongated coupling member extends along the longitudinal axis, centrally within the set of wrist links. The elongated coupling member is positioned to apply a central restoring tension to compress the first link and second link.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a set of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments, however, any of the links, tool members, beams, shafts, cables, or components described herein can be monolithically constructed.

Although the longitudinal axis of the engagement protrusion 2560 is shown as being orthogonal to the longitudinal axis of the engagement recess 2580, in other embodiments, the engagement protrusion 2560 can oriented at any angle relative to the engagement recess 2580. For example, in some embodiments, the longitudinal axis of the engagement protrusion 2560 can oriented at 45 degrees from the longitudinal axis of the engagement recess 2580. In this manner, each joint disk 2510 can be oriented 45 degrees from its adjacent joint disk about the longitudinal axis A_{LO1}.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1-45. (canceled)

46. A medical instrument, comprising:
an instrument shaft defining a longitudinal axis;
a proximal disk coupled to a distal end of the instrument shaft;
a distal disk coupled to an end effector of the medical instrument;
a joint disk positioned between and in axial alignment with the proximal disk and the distal disk, the joint disk defining a portion of a central passage axially aligned with the longitudinal axis, each of the joint disk and the proximal disk having a distal face, each of the joint disk and the distal disk having a proximal face, wherein:
each distal face includes an engagement protrusion,
each proximal face includes an engagement recess,
each engagement recess is sized to receive a corresponding engagement protrusion,
the engagement protrusion of the joint disk has a length that is orthogonal to a length of engagement recess of the joint disk; and
a coupling member within the central passage and extending between the proximal disk and the distal disk, the coupling member being fixedly coupled to the proximal disk and to the distal disk, the coupling member having an absence of coupling with the joint disk.

47. The medical instrument of claim 46, wherein:
the joint disk defines a distal reference plane orthogonal to the longitudinal axis;
the engagement protrusion of the joint disk extends longitudinally outward from the distal reference plane;
the joint disk defines a proximal reference plane orthogonal to the longitudinal axis and parallel to the distal reference plane;
a longitudinal separation between the distal reference plane and the proximal reference plane corresponds to a maximal thickness of the joint disk; and
a depth of the engagement recess of the joint disk along the longitudinal axis relative to the proximal reference plane has a ratio relative to the maximal thickness of between 1:1.4 and 1:1.5.

48. The medical instrument of claim 46, wherein:
each engagement recess defines a proximal passage opening of the central passage;
each engagement protrusion defines a distal passage opening;
a diameter of each distal passage opening is greater than a diameter of each proximal passage opening; and
each proximal passage opening is sized to maintain the coupling member in alignment with a neutral axis extending between the proximal disk and the distal disk.

49. The medical instrument of claim 48, wherein:
a ratio of the diameter of the proximal passage opening to the diameter of the distal passage opening corresponds to a maximum range of angular movement between at least one of the proximal disk and the joint disk, and the joint disk and the distal disk.

50. The medical instrument of claim 46, wherein:
the joint disk includes a central passage wall extending between a proximal passage opening and a distal passage opening of the joint disk;
the central passage wall has a taper angle that extends away from the longitudinal axis in a distal direction; and
the taper angle is proportional to a maximum range of angular movement between at least one of the proximal disk and the joint disk, and the joint disk and the distal disk.

51. The medical instrument of claim 46, wherein:
the coupling member is pretensioned via the fixed coupling to the proximal disk and the distal disk; and
the coupling member is a super elastic material.

52. The medical instrument of claim 46, wherein:
each distal face defines a distal contact face;
each proximal face defines a proximal contact face; and
the positioning of the distal contact face relative to an opposing proximal contact face defines a range of motion limit for at least one of the proximal disk and the joint disk, and the joint disk and the distal disk about a pivot axis defined by the engagement protrusion and the engagement recess.

53. The medical instrument of claim 52, wherein:
the distal contact face is in contact with the opposing proximal contact face on a condition that at least one of the proximal disk and the joint disk, and the joint disk and the distal disk are at a maximal flexion angle.

54. The medical instrument of claim 52, wherein:
the distal contact face of the joint disk has an angle relative to a distal reference plane of the joint disk that is one half of a maximal flexion angle of the joint disk relative to the distal disk; and
the proximal contact face of the joint disk has an angle relative to a proximal reference plane of the joint disk that is one half of a maximum flexion angle of the joint disk relative to the proximal disk.

55. A joint disk of a multi-axis joint structure of a medical instrument, the joint disk comprising:
a circular body positioned orthogonally to a longitudinal axis of the joint disk, the circular body defining a central passage coaxial with the longitudinal axis of the joint disk, the central passage being configured to receive a coupling member;
a distal face of the circular body, the distal face including a first distal contact face, a second distal contact face, and an engagement protrusion positioned therebetween, the engagement protrusion being a cylindrical segment having a length bisected by the longitudinal axis of the joint disk and extending parallel to a first lateral axis; and
a proximal face of the circular body positioned longitudinally opposite the distal face, the proximal face including a first proximal contact face, a second proximal contact face, and an engagement recess positioned therebetween, the engagement recess being a cylindrical segment having a length bisected by the longitudinal axis of the joint disk and extending parallel to a second lateral axis orthogonal to the first lateral axis.

56. The joint disk of claim 55, wherein:
the distal face includes a first recessed portion extending parallel to the length of the engagement protrusion between the engagement protrusion and the first distal contact face; and
the distal face includes a second recessed portion extending parallel to the length of the engagement protrusion between engagement protrusion and the second distal contact face.

57. The joint disk of claim 55, wherein:
the distal face includes a transition portion extending radially outward from the engagement protrusion between the first distal contact face and the second distal contact face;
the transition portion extends across a width of the engagement protrusion; and
the transition portion is shaped to preclude contact with an adjacent component of the multi-axis joint structure on a condition that the joint disk is pivoted about the first lateral axis.

58. The joint disk of claim 55, wherein:
the length of the engagement protrusion extends along an axial centerline of the cylindrical segment;
the axial centerline lies on a distal reference plane defined by the joint disk orthogonal to the longitudinal axis;
the first distal contact face defines a first distal contact plane;
the first distal contact plane includes the axial centerline of the engagement protrusion and is inclined relative to the distal reference plane;
the second distal contact face defines a second distal contact plane; and
the second distal contact plane includes the axial centerline, intersects the first distal contact plane, and is inclined relative to the distal reference plane.

59. The joint disk of claim 58, wherein:
a magnitude of the inclination of the first distal contact plane and the magnitude of the inclination of the second distal contact plane relative to a distal reference plane are equal.

60. The joint disk of claim 58, wherein:
the circular body defines a taper angle for the central passage; and
the taper angle has a magnitude that is twice the magnitude of the inclination of the first distal contact plane relative to the distal reference plane.

61. The joint disk of claim 55, wherein:
the length of the engagement recess extends along an axial centerline of the cylindrical segment;
the axial centerline lies on a proximal reference plane defined by the joint disk orthogonal to the longitudinal axis and proximal to the circular body;
the first proximal contact face defines a first proximal contact plane;
the first proximal contact plane includes the axial centerline of the engagement recess and is inclined relative to the proximal reference plane;
the second proximal contact face defines a second proximal contact plane; and
the second proximal contact plane includes the axial centerline, intersects the first proximal contact plane, and is inclined relative to the proximal reference plane.

62. The joint disk of claim 61, wherein:
a magnitude of the inclination of the first proximal contact plane and the magnitude of the inclination of the second proximal contact plane relative to the proximal reference plane are equal.

63. The joint disk of claim 61, wherein:
the first distal contact face defines a first distal contact plane having an inclination relative to a distal reference plane defined by the joint disk;
the second distal contact face defines a second distal contact plane having an inclination relative to the distal reference plane;
a magnitude of the inclination of the first distal contact plane and the magnitude of the inclination of the second distal contact plane relative to the distal reference plane are equal; and
the magnitude of the inclination of the first distal contact plane and the magnitude of the inclination of the second distal contact plane are equal to the magnitude of the inclination of the first proximal contact plane and the magnitude of the inclination of the second proximal contact plane.

64. The joint disk of claim 55, wherein:
the joint disk includes a central passage wall extending between a proximal passage opening and a distal passage opening of the joint disk;
the central passage wall is curvilinear in a longitudinal direction; and
each of the distal passage opening and the proximal passage opening include one of a chamfer region or a rounded over region.

65. A medical instrument comprising:
an instrument body elongated along a longitudinal axis between a distal end and a proximal end;
an end effector coupled to the distal end;
an articulatable wrist coupled between the instrument and the instrument body, the articulatable wrist being elongated and configured to curve along a first plane, a second plane transverse to the first plane, and in between the first and second plane without imparting rotation to the instrument body, the articulatable wrist comprising a plurality of wrist links arranged in a stack extending along the longitudinal axis, the plurality of wrist links comprising a first link rotationally engaged with and laterally constrained by a second link;
a plurality of control cables extending along the longitudinal axis, peripherally within the plurality of wrist links, the plurality of control cables arranged to selectively apply a peripheral tension to the first link and second link to cause the articulatable wrist to curve; and
an elongated coupling member extending along the longitudinal axis, centrally within the plurality of wrist links, the elongated coupling member applying a central restoring tension to compress the first link and second link.
